SEQUENCE LISTING (2 sequences)

SEQ ID NO: 1 - protein, 17 aa, Artificial Sequence
  Description of Artificial Sequence Synthetic peptide
AWSFRVSYRGISYRRSR

SEQ ID NO: 2 - protein, 18 aa, Artificial Sequence
  Description of Artificial Sequence Synthetic peptide
RGGRLSYSXXXFSTSTGR

(19) United States
(12) Patent Application Publication
(10) Pub. No.: US 2005/0159360 A1
(43) Pub. Date: Jul. 21, 2005

(54) Compositions for the transport of therapeutic molecules into the lungs and use thereof for the treatment of lung cancers and pulmonary diseases

(75) Inventors: Jamal Temsamani, Nimes (FR); Anthony R. Rees, Lund (SE); Christophe Rousselle, Paris (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: SYNT:EM, a corporation of France, Nimes (FR)

(21) Appl. No.: 11/016,318

(22) Filed: Dec. 17, 2004

Related U.S. Application Data
(63) Continuation of application No. PCT/FR03/01864, filed on Jun. 18, 2003.

(30) Foreign Application Priority Data
Jun. 18, 2002 (FR) FR 02/07493

(51) Int. Cl. A61K 38/10; A61K 31/704; A61K 31/337

(52) U.S. Cl. 514/13; 514/34; 514/449

(57) ABSTRACT

A pharmaceutical composition including at least one therapeutic molecule effective for treating lung cancers or pulmonary diseases; and at least one peptide vector that augments bioavailability of the molecule in a patient's lungs selected from the group consisting of Ala-Trp-Ser-Phe-Arg-Val-Ser-Tyr-Arg-Gly-Ile-Ser-Tyr-Arg-Arg-Ser-Arg (SynB4) (SEQ ID No. 1), and Arg-GLy-Gly-Arg-Leu-Ser-Tyr-Ser-Cit-Cit-Cit-Phe-Ser-Thr-Ser-Thr-Gly-Arg (SynB6) (SEQ ID No. 2).

### RELATED APPLICATION

This is a continuation of International Application No. PCT/FR03/01864, with an international filing date of Jun. 18, 2003 (WO 03/105907, published Dec. 24, 2003), which is based on French Patent Application No. 02/07493, filed Jun. 18, 2002.

### FIELD OF THE INVENTION

This invention pertains to the use of peptide vectors for the transport of active substances intended for the treatment of diseases that affect the lungs such as lung cancers and respiratory diseases. The invention also relates to compounds and the pharmaceutical compositions containing them that are useful for the treatment of lung cancers and pulmonary diseases.

### BACKGROUND

An inevitable consequence of the increase in smokers, lung cancer has become the leading cause of death from cancer in the United States and is about to follow the same path in France. Globally, the five-year cancer survival rate barely exceeds 10%. Although surgery alone can cure the rare cases of small-cell cancers or when the tumor is discovered at a very limited stage, hopes rest on the combination of different treatments for more evolved tumors. By diminishing the intensity of the symptoms, chemotherapy improves the quality of life of patients with lung cancer. However, despite the progress achieved in the modalities of administration of therapeutic combinations, the treatment of these tumors remains very difficult.

The treatment of lung cancers with chemotherapy is limited principally by the toxicity and low bioavailability of anticancer agents. Consequently, most anticancer agents must be administered in very high doses to reach the lungs, but at the price of notable side effects.

Bronchitis and emphysema are also diseases associated with smoking. Bronchitis, like numerous pulmonary and respiratory diseases such as pneumonia and cystic fibrosis, is often accompanied by the accumulation of secretions that can induce respiratory distress and even in certain cases lead to death.

Cystic fibrosis (or mucoviscidosis) is an autosomal and recessive hereditary genetic disease affecting children. Mutation of the CFTR gene responsible for the transport of chloride ions leads to the obstruction of the respiratory pathways by accumulation of mucus.

These diseases including emphysema are aggravated by the invasion of bacteria colonies promoted by the accumulation of secretions in the respiratory pathways.

Products intended to aid the protein CFTR reach the surface of cells or products capable of stimulating or impeding other ionic channels, or the introduction of deficient fatty acids into the cells, or the administration of antibiotics intended to combat the bacteria have to date yielded disappointing results. In fact, the toxicity to the organism of the products administered in a sufficient quantity to reach the lungs considerably limits their use.

Independent of the diseases cited above, there are numerous bronchopulmonary diseases of viral and bacterial origin. Many of these diseases have become very grave and difficult to combat because of the resurgence of bacterial strains resistant to antibiotics. As examples, diseases such as tuberculosis caused by Mycobacterium tuberculosis and pneumonia whose pathogenic agents have for origin essentially the opportunistic bacteria of the genus Pseudomonas.

Employment of new compounds and new methods for treating pulmonary and respiratory diseases would therefore constitute a significant advance in the art. In that regard, we previously demonstrated that linear peptide vectors, such as the linear peptides derived from natural peptides such as protegrin and tachyplesin, can transport active molecules through the biological membranes and improve the pharmacological properties of these molecules. The studies and results pertaining to these linear peptides and their use as vectors of active molecules were described in application FR 98/15074, filed on Nov. 30, 1998, and patent FR 99/02938, filed on Nov. 26, 1999.

### SUMMARY OF THE INVENTION

This invention relates to a pharmaceutical composition including at least one therapeutic molecule effective for treating lung cancers or pulmonary diseases; and at least one peptide vector that augments bioavailability of the molecule in a patient's lungs selected from the group consisting of Ala-Trp-Ser-Phe-Arg-Val-Ser-Tyr-Arg-Gly-Ile-Ser-Tyr-Arg-Arg-Ser-Arg (SynB4) (SEQ ID No. 1), and Arg-GLy-Gly-Arg-Leu-Ser-Tyr-Ser-Cit-Cit-Cit-Phe-Ser-Thr-Ser-Thr-Gly-Arg (SynB6) (SEQ ID No. 2).

This invention also relates to a method of treating lung cancers or pulmonary diseases in a patient including administering a therapeutically effective amount of a pharmaceutical composition including at least one therapeutic molecule effective for treating lung cancers or pulmonary diseases; and at least one peptide vector that augments bioavailability of the molecule in a patient's lungs selected from the group consisting of Ala-Trp-Ser-Phe-Arg-Val-Ser-Tyr-Arg-Gly-Ile-Ser-Tyr-Arg-Arg-Ser-Arg (SynB4) (SEQ ID No. 1), and Arg-GLy-Gly-Arg-Leu-Ser-Tyr-Ser-Cit-Cit-Cit-Phe-Ser-Thr-Ser-Thr-Gly-Arg (SynB6) (SEQ ID No. 2) to the patient.

This invention further relates to a method of preventing lung cancers or pulmonary diseases in a patient including administering a therapeutically effective amount of a pharmaceutical composition including at least one therapeutic molecule effective for treating lung cancers or pulmonary diseases; and at least one peptide vector that augments bioavailability of the molecule in a patient's lungs selected from the group consisting of Ala-Trp-Ser-Phe-Arg-Val-Ser-Tyr-Arg-Gly-Ile-Ser-Tyr-Arg-Arg-Ser-Arg (SynB4) (SEQ ID No. 1), and Arg-GLy-Gly-Arg-Leu-Ser-Tyr-Ser-Cit-Cit-Cit-Phe-Ser-Thr-Ser-Thr-Gly-Arg (SynB6) (SEQ ID No. 2) to the patient.

### BRIEF DESCRIPTION OF THE DRAWINGS

Other advantages and characteristics of the invention will become apparent from the examples below pertaining to the preparation of compounds constituted by doxorubicin and linear peptides. Reference will be made to the attached drawings in which:

FIG. 1 schematically represents the chemical synthesis of a vectorized compound of doxorubicin, and

FIG. 2 illustrates a comparison of the pharmacokinetics/biodistribution of free doxorubicin and doxorubicin coupled to SynB4 and SynB6.

### DETAILED DESCRIPTION

We have now discovered amino acid sequences capable of serving as an internalization vector and specifically addressing active substances in a specific organ, i.e., the lung. In particular, we discovered the following peptide sequences:

(SEQ ID No. 1)

Ala-Trp-Ser-Phe-Arg-Val-Ser-Tyr-Arg-Gly-Ile-Ser-
Tyr-Arg-Arg-Ser-Arg (Synb4),

(SEQ ID No. 2)

Arg-Gly-Gly-Arg-Leu-Ser-Tyr-Ser-Cit-Cit-Cit-Phe-
Ser-Thr-Ser-Thr-Gly-Arg (SynB6)

are capable of specifically addressing the active substances at the level of the lung and enabling the internalization of the active substances in this organ.

This invention thus relates to a compound including at least one therapeutic molecule adapted for the treatment of lung cancers or pulmonary diseases and at least one peptide vector capable of augmenting the bioavailability of the molecule at the level of the lungs. The invention includes, most particularly, a peptide vector capable of augmenting the bioavailability of the molecule at the level of the lungs, the peptide being selected from:

(SEQ ID No. 1)

Ala-Trp-Ser-Phe-Arg-Val-Ser-Tyr-Arg-Gly-Ile-Ser-
Tyr-Arg-Arg-Ser-Arg (SynB4),

(SEQ ID No. 2)

Arg-Gly-Gly-Arg-Leu-Ser-Tyr-Ser-Cit-Cit-Cit-Phe-
Ser-Thr-Ser-Thr-Gly-Arg
(SynB6).

Non-limiting examples of therapeutic molecules intended for the treatment of lung cancers employed in the compounds of the invention include anticancer agents such as paclitaxel, doxorubicin and the like. Non-limiting examples of therapeutic molecules intended for the treatment of pulmonary diseases employed in the compounds of the invention include antibiotics, antimicrobial peptides and the like. As nonlimitative examples, the antibiotics can be benzylpenicillin, erythromycin, amoxicillin and the like. The antimicrobial peptides are such as the human tracheal antimicrobial peptide (hTAP) and the peptides described in U.S. Pat. Nos. 5,202,420 and 5,459,235. These examples are only presented for indicative purposes and those of ordinary skill in the art can employ various types of therapeutic molecules intended for the treatment of pulmonary diseases.

In the compounds of the invention, the therapeutic molecules intended for the treatment of lung cancers or pulmonary diseases can be linked directly or indirectly to the peptide vectors.

The link between the therapeutic molecule intended for the treatment of lung cancers or pulmonary diseases and the linear peptide vector may be selected from among a covalent bond, a hydrophobic bond, an ionic bond, a cleavable bond or a noncleavable bond in the physiological media or the interior of the cells.

This link can be implemented by the intermediary of a linker arm between the therapeutic molecule and the peptide vector at the level of a functional group that is naturally present or is introduced either on the peptide or on the therapeutic molecule, or on both. This linker arm, if it is present, should be acceptable taking into account the chemical nature and the size both of the peptide and the therapeutic molecule. Nonexhaustive examples of linker arms that can be used include bifunctional or multifunctional agents containing an alkyl, aryl, alkylaryl or peptide groups, esters, amides, amines, alkyl or aryl or alkylaryl aldehydes or acids, anhydrides, sulfhydryls or carboxyl groups such as the derivatives of benzoic maleimilic acid, propionic maleimilic acid and succinimidyl derivatives, derivative groups of cyanogen bromide or chloride, carbonyldiimidazole, esters, phosgene, esters of succinimide or sulfonic halides.

The following can be cited as functional groups: —OH, —SH, —COOH or —NH₂. Thus, the therapeutic molecule can be linked by covalent bonds at the level of the N-terminal or C-terminal ends or at the level of the lateral chains of the peptide.

The invention also relates to compounds comprising a therapeutic molecule intended for the treatment of lung cancers or pulmonary diseases linked to multiple peptide vectors capable of augmenting the bioavailability of the molecule at the level of the lungs or to multiple identical or different therapeutic molecules intended for the treatment of lung cancers or pulmonary diseases linked to a peptide vector capable of augmenting the bioavailability of the molecule at the level of the lungs. The invention includes polymers of such compounds as well.

The invention further relates to a method for the treatment or the prevention of lung cancers and pulmonary diseases comprising administering to a subject suffering from such a disease an effective amount of a compound as described above. The invention thus pertains to a pharmaceutical composition for the treatment of lung cancers and pulmonary diseases comprising as active agent at least one compound as described above.

The pharmaceutical composition preferably is in a form suitable for administration via the systemic route, the parenteral route, the oral route, the rectal route, the nasal route, the transdermal route, the pulmonary route or the central route.

As previously stated, the linear peptides are remarkable in that they are capable of transporting in a selective manner the therapeutic molecule into the lungs after systemic administration and thus to enable delivery of a large amount of active substance at the level of the site of action, thus making it possible to increase their efficacy and reduce the side effects.

The invention thus relates to the use of a linear peptide as defined above a drug intended for the treatment and/or prevention of lung cancers or pulmonary diseases, the peptide being linked in the drug to at least one active molecule for transporting the active molecule in a specific manner into the lungs.

I—Chemical Synthesis of Vectorized Doxorubicin

1) Synthesis of the Peptide Vectors

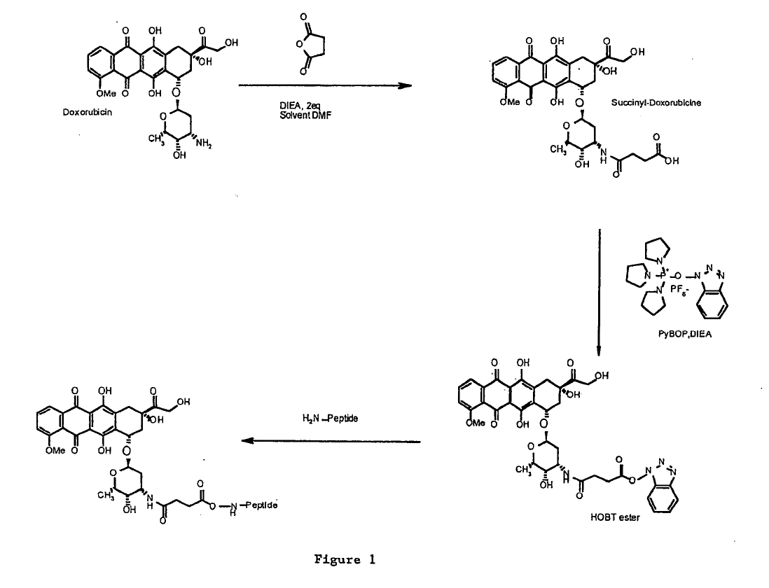

The peptides synB4 of sequence Ala-Trp-Ser-Phe-Arg-Val-Ser-Tyr-Arg-Gly-Ile-Ser-Tyr-Arg-Arg-Ser-Arg (SEQ ID No. 1) and SynB6 of sequence Arg-Gly-Gly-Arg-Leu-Ser-Tyr-Ser-Cit-Cit-Cit-Phe-Ser-Thr-Ser-Thr-Gly-Arg (SEQ ID No. 2) were assembled on solid phase according to an Fmoc/tBu strategy, cleaved and deprotected by trifluoroacetic acid, then purified by preparative high pressure chromatography in inverse phase and lyophilized. FIG. 1 presents a diagram of this preparation method. Their purity (>95%) and their identity were confirmed by analytic HPLC and by mass spectrometry.

2) Coupling of Doxorubicin on the Peptide Vectors

a) Preparation of the Peptides Coupled to Doxorubicin

The coupling of doxorubicin on the peptides via the intermediary of the succinic link was performed in 3 steps.

To doxorubicin hydrochloride (1 eq.) dissolved in dimethylformamide (DMF) in the presence of diisopropylethylamine (DIEA, 2 eq.) was added succinic anhydride (1.1 eq., dissolved in DMF). After incubation of 20 minutes at ambient temperature, the thereby formed doxorubicin hemisuccinate was then activated by addition of PyBOP benzotriazol-1-yl-oxopyrrolidinephosphonium hexafluorophosphate (1.1 eq.) in DMF and DIEA (2 eq.). This second reaction mixture was incubated for 20 minutes. The peptide (1.2 eq. in DMF) was then added to the reaction mixture and coupled spontaneously on the doxorubicin hemisuccinate activated during a supplementary incubation of 20 minutes.

The coupling product was then purified on preparative HPLC (high pressure liquid chromatography) then lyophilized.

Each of the steps as well as the final product were checked with analytic HPLC and mass spectrometry.

b) Radioactive Tagging of the Peptides Coupled to Doxorubicin

Preparation and purification of the radioactive products was performed as described above except that the doxorubicin was replaced by radioactive doxorubicin ([¹⁴C]-doxorubicin (specific activity 55 Ci/mmol, 2.04 TBq/mol; Amersham, Les Ulis, France)). The specific activity of the products dox-SynB4 and dox-SynB6 at the end of the reactions was 55 Ci/mmol (i.e., 2.04 TBq/mol) and their radiochemical purity was >98%.

II—Compounds Tested

The compounds tested are presented in table 1 below.

TABLE 1
Compound

| | |
|---|---|
| Compound 1 (dox) | doxorubicin |
| Compound 2 | AWSFRVSYRGISYRRSR-succ-dox |
| (SynB4-dox) | |
| Compound 3 | RGGRLSYS-Cit-Cit-Cit-FSTSTGR-succ-dox |
| (SynB6-dox) | |

III—Intravenous Injections

Mice were injected via the intravenous route with vectorized doxorubicin (compounds 2 and 3) or doxorubicin alone (compound 1) at a dose of 1 mg/kg (doxorubicin equivalent). About 0.6-1 microcurie was injected per animal. The doxorubicin was tagged with carbon 14 (specific activity 55 mCi/mmol). After the indicated time periods (1, 5, 15, 30, 60 minutes), the mice were sacrificed. The organs (lungs, liver, brain, kidneys, etc.) and the plasma were then collected and counted. The quantity of radioactivity in each organ was then expressed as quantity of product per gram of organ. In this study, five mice were used for each time period.

IV—Results

After injection of the doxorubicin or the vectorized doxorubicin, we compared the pharmacokinetics/biodistribution of the products in the plasma and the different organs. The quantity of each product was expressed as percentage of product per organ.

TABLE 2
Percentage of doxorubicin (compound 1) after intravenous injection

| Time | | | | | | |
|---|---|---|---|---|---|---|
| (minutes) | Plasma | Brain | Heart | Lungs | Kidneys | Liver |
| 1 | 1.84 | 0.06 | 0.58 | 1.30 | 3.37 | 17.81 |
| 5 | 0.53 | 0.03 | 0.52 | 1.24 | 5.58 | 23.02 |
| 15 | 0.34 | 0.03 | 0.54 | 1.14 | 5.24 | 20.22 |
| 30 | 0.19 | 0.02 | 0.45 | 1.03 | 4.48 | 19.70 |
| 60 | 0.18 | 0.02 | 0.36 | 0.85 | 2.43 | 17.80 |

TABLE 3
Percentage of dox-SynB4 (compound 2) after intravenous injection

| Time | | | | | | |
|---|---|---|---|---|---|---|
| (minutes) | Plasma | Brain | Heart | Lungs | Kidneys | Liver |
| 1 | 1.07 | 0.07 | 0.47 | 33.3 | 2.3 | 18.4 |
| 5 | 0.7 | 0.05 | 0.41 | 46.9 | 2.0 | 25.7 |
| 15 | 0.32 | 0.04 | 0.33 | 16.0 | 1.3 | 35.7 |
| 30 | 0.19 | 0.04 | 0.26 | 11.0 | 1.0 | 35.1 |
| 60 | 0.12 | 0.04 | 0.35 | 16.4 | 1.1 | 37.2 |

TABLE 4
Percentage of dox-SynB6 (compound 3) after intravenous injection

| Time | | | | | | |
|---|---|---|---|---|---|---|
| (minutes) | Plasma | Brain | Heart | Lungs | Kidneys | Liver |
| 1 | 1.60 | 0.20 | 0.50 | 60.0 | 2.6 | 10.9 |
| 5 | 0.80 | 0.08 | 0.24 | 68.5 | 2.2 | 16.1 |
| 15 | 0.46 | 0.07 | 0.15 | 46.6 | 2.1 | 15.7 |
| 30 | 0.35 | 0.06 | 0.15 | 50.6 | 1.8 | 21.8 |
| 60 | 0.18 | 0.07 | 0.11 | 38.2 | 1.8 | 23.9 |

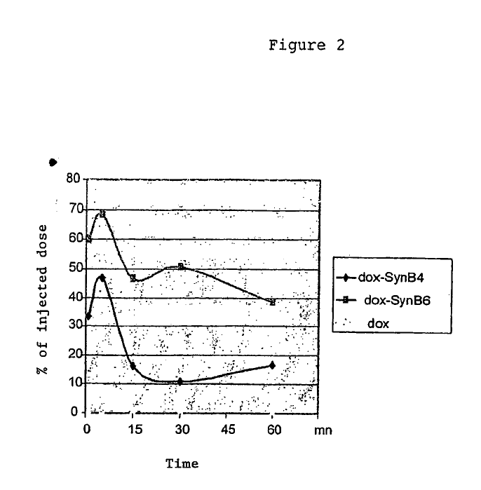

These results demonstrate that the coupling of doxorubicin with SynB6 or SynB4 significantly improves its biodistribution in the lungs. This augmentation is specific to the lungs since the biodistribution in the other organs did not change significantly after vectorization. FIG. 2 presents a comparison of the biodistribution in the lungs of free doxorubicin and vectorized doxorubicin.

1. A pharmaceutical composition comprising at least one therapeutic molecule effective for treating lung cancers or pulmonary diseases; and at least one peptide vector that augments bioavailability of the molecule in a patient's lungs selected from the group consisting of:
(SEQ ID No. 1)
Ala-Trp-Ser-Phe-Arg-Val-Ser-Tyr-Arg-Gly-Ile-Ser-
Tyr-Arg-Arg-Ser-Arg (SynB4),
and
(SEQ ID No. 2)
Arg-GLy-Gly-Arg-Leu-Ser-Tyr-Ser-Cit-Cit-Cit-Phe-
Ser-Thr-Ser-Thr-Gly-Arg (SynB6).

2. The composition according to claim 1, wherein the therapeutic molecule is an anticancer agent.

3. The composition according to claim 2, wherein the anticancer agent is paclitaxel or doxorubicin.

4. The composition according to claim 1, wherein the therapeutic molecule is an antibiotic or an antimicrobial peptide.

5. The composition according to claim 1, wherein the therapeutic molecule is linked directly or indirectly to the peptide vector.

6. The composition according to claim 4, wherein the link is a covalent bond, a hydrophobic bond, an ionic bond, a cleavable bond or a noncleavable bond in the physiological media or in the interior of the cells.

7. The composition according to claim 5, wherein the link has a linker arm between the therapeutic molecule and the peptide vector at the level of a functional group naturally present or introduced either on the peptide or on the therapeutic molecule, or on both.

8. The composition according to claim 6, wherein the link has a linker arm between the therapeutic molecule and the peptide vector at the level of a functional group naturally present or introduced either on the peptide or on the therapeutic molecule, or on both.

9. The composition according to claim 7, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.

10. The composition according to claim 8, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.

11. The composition according to claim 9, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

12. The composition according to claim 10, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

13. A method of treating lung cancers or pulmonary diseases in a patient comprising administering a therapeutically effective amount of a pharmaceutical composition comprising at least one therapeutic molecule effective for treating lung cancers or pulmonary diseases; and at least one peptide vector that augments bioavailability of the molecule in a patient's lungs selected from the group consisting of:
(SEQ ID No. 1)
Ala-Trp-Ser-Phe-Arg-Val-Ser-Tyr-Arg-Gly-Ile-Ser-
Tyr-Arg-Arg-Ser-Arg (SynB4),
and
(SEQ ID No. 2)
Arg-GLy-Gly-Arg-Leu-Ser-Tyr-Ser-Cit-Cit-Cit-Phe-
Ser-Thr-Ser-Thr-Gly-Arg (SynB6)
to the patient.

14. The method according to claim 13, wherein the therapeutic molecule is an anticancer agent.

15. The method according to claim 14, wherein the anticancer agent is paclitaxel or doxorubicin.

16. The method according to claim 13, wherein the therapeutic molecule is an antibiotic or an antimicrobial peptide.

17. The method according to claim 13, wherein the therapeutic molecule is linked directly or indirectly to the peptide vector.

18. The method according to claim 17, wherein the link is a covalent bond, a hydrophobic bond, an ionic bond, a cleavable bond or a noncleavable bond in the physiological media or the interior of the cells.

19. The method according to claim 17, wherein the link has a linker arm between the active molecule and the peptide vector at the level of a functional group naturally present or introduced either on the peptide or on the molecule, or on both.

20. The method according to claim 13, wherein the link has a linker arm between the active molecule and the peptide vector at the level of a functional group naturally present or introduced either on the peptide or on the molecule, or on both.

21. The method according to claim 19, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.

22. The method according to claim 20, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.

23. The composition according to claim 21, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

24. The composition according to claim 22, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

25. A method of preventing lung cancers or pulmonary diseases in a patient comprising administering a therapeutically effective amount of a pharmaceutical composition comprising at least one therapeutic molecule effective for treating lung cancers or pulmonary diseases; and at least one peptide vector that augments bioavailability of the molecule in a patient's lungs selected from the group consisting of:
(SEQ ID No. 1)
Ala-Trp-Ser-Phe-Arg-Val-Ser-Tyr-Arg-Gly-Ile-Ser-
Tyr-Arg-Arg-Ser-Arg (SynB4),
and
(SEQ ID No. 2)
Arg-GLy-Gly-Arg-Leu-Ser-Tyr-Ser-Cit-Cit-Cit-Phe-
Ser-Thr-Ser-Thr-Gly-Arg (SynB6)
to the patient.

26. The method according to claim 25, wherein the therapeutic molecule is an anticancer agent.

27. The method according to claim 26, wherein the anticancer agent is paclitaxel or doxorubicin.

28. The method according to claim 25, wherein the therapeutic molecule is an antibiotic or an antimicrobial peptide.

29. The method according to claim 25, wherein the therapeutic molecule is linked directly or indirectly to the peptide vector.

30. The method according to claim 29, wherein the link is a covalent bond, a hydrophobic bond, an ionic bond, a cleavable bond or a noncleavable bond in the physiological media or the interior of the cells.

31. The method according to claim 29, wherein the link has a linker arm between the active molecule and the peptide vector at the level of a functional group naturally present or introduced either on the peptide or on the molecule, or on both.

32. The method according to claim 25, wherein the link has a linker arm between the active molecule and the peptide vector at the level of a functional group naturally present or introduced either on the peptide or on the molecule, or on both.

33. The composition according to claim 31, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

34. The composition according to claim 32, wherein the linker arm is a derivative of benzoic maleimilic acid, propionic maleimilic acid and a succinimidyl derivative, a derivative group of cyanogens, bromide or chloride, carbonyldiimidazole, esters, phosgene, or esters of succinimide or sulfonic halides.

35. The method according to claim 31, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.

36. The method according to claim 32, wherein the linker arm is a bifunctional or multifunctional agent containing an alkyl, aryl, alkylaryl or peptide groups, esters, amides, amines, alkyl or aryl or alkylaryl aldehydes or acids, anhydrides, sulfhydryls or carboxyl groups.
